(19) United States
(12) Patent Application Publication
(10) Pub. No.: US 2014/0025012 A1
(43) Pub. Date: Jan. 23, 2014

(54) Clinician Hand Support Structure

(71) Applicant: Brett Coval, Denver, CO (US)

(72) Inventor: Brett Coval, Denver, CO (US)

(21) Appl. No.: 13/554,534

(22) Filed: Jul. 20, 2012

(51) Int. Cl. A61M 5/31

(52) U.S. Cl. 604/187

(57) ABSTRACT

A clinician's hand support structure provides an ergometric grasp of a syringe housing, the support structure includes a surrounding sidewall about a longitudinal axis, the sidewall having first and second end portions, the sidewall also having an outer surface and an inner surface that defines a sidewall interior. Further, the inner surface having a portion with a frustroconical shape that is smaller adjacent to the first end portion and larger adjacent to the second end portion. Also, the sidewall has an elongated aperture therethrough, wherein the sidewall inner surface partially creates a frictional encasement of the syringe housing. Further, included is an extension element having a proximal and distal portion wherein the extension element is positioned to extend radially outward from the longitudinal axis, operationally the distal portion nests in a palm of a clinician's hand and the sidewall outer surface is grasped by a finger of the clinician.

### RELATED PATENT APPLICATIONS

There are no related patent applications.

### TECHNICAL FIELD

The present invention relates generally to a structure for providing a more ergometric grasp of a hand tool. More specifically, the present invention relates to the field of removably engagable structures that enable more secure, comfortable, and less fatiguing manual grasping of specific hand tools during their use.

### BACKGROUND OF INVENTION

Frequently, hand tools are designed for a particular function that dictates the external configuration of the hand tool, whether it be for size, weight, materials of construction, control placement, strength, disbursement of a fluid, aiming of a light, and the like. Because of this, the manual hand grip of the tool may not be optimum for the user, being especially important for a user who needs to use, i.e. manually grip the tool for extended periods of time or alternatively to for the user to engage in a high number of repeated uses, i.e. grasping and un-grasping the tool many times through the day. In these situations a comfortable and non-fatiguing grip is more important, however, the tool may not have included in its design an ergometrically optimized hand grip, thus resulting in the need for a removably engagable manual hand grip that engages the tool and provides an ergometric hand grip for comfort and less hand fatigue.

In the prior art, looking at United States Patent Application Number 2012/0041388 to Blomquist, disclosed is a handle device for a syringe, comprising a handle body, with the body having a lateral aperture therethrough, thereby forming a first end of the handle body. Also in Blomquist, being opposite of the aperture, a second end of the handle body, an index finger support region at the first end of the handle body, with the index finger support region being adapted to receive an index finger of an operator. Further in Blomquist, a syringe attachment region is on the handle body that is adapted to attach the handle body to a syringe, and an other-finger support region at the second end of the handle body, the other-finger support region is adapted to receive at least one of a middle finger, a ring finger, and a pinkie of the operator, whereby an operator or user can firmly grasp an assembly of the handle device and a syringe by resting an index finger on the index finger support region, at least one other finger on the other-finger support region, and a thumb on a plunger of the syringe.

Continuing, in the prior art in U.S. Pat. No. 6,183,400 to Pope, disclosed is an attachment or accessory for exercise devices such as barbells, dumbbells, weight training machines, or any device utilizing a cylindrical bar. In Pope, the accessory or attachment is intended for strengthening a user's targeted muscle without the necessity of a forceful grip and provides a safer, more effective, and efficient workout. The device in Pope is made up of two parts, which are hinged together, to form a grasping area of 16 square inches as opposed to 4 square inches of grasping area on a regular one inch diameter bar. The unit in Pope is secured to the bar with a latch and there is a palm heel to prevent the unit from rotating and to help keep the shaft approximately 90 degrees to the hand. The strap in Pope uniformly spreads the weight of the exercise device over the back of the hand reducing the need of forceful grip for lifting the weights when the hand is over the weight.

Further, in the prior art in U.S. Pat. No. 6,197,006 to Wiklund disclosed is an ergonomic handle for a syringe intended for puncturing blood vessel walls and establishing blood path connections, wherein the typical syringe comprises a tube body and a needle body. The handle in Wiklund prevents unintended displacements between the tube body and the needle body and permits placement of the thumb and forefinger at the side and the tip of the puncture needle for more precision in use.

Next, in the prior art U.S. Pat. No. 7,840,964 to Vidal discloses a hand-held instrument has a business end and a handle attached to the business end with the handle having a gripping portion and a longitudinal axis. A knob in Vidal is provided on the gripping portion of the handle to facilitate the positive grip wielding of the hand-held instrument with the knob being disposed such that it projects away from the handle in a direction transverse to the longitudinal axis of the handle. In Vidal, a preferred, but not required embodiment, the knob is removably attached to the handle by a quick release attachment device and in a typical, but not required embodiment, the quick release attachment device includes a male connection pin disposed within the knob and a female receptor disposed within the hand tool.

Continuing, in the prior art for U.S. Pat. No. 7,673,770 to Summerfield, disclosed is a flexible soft sleeve for enhancing a user's grasp of the handles and handholds of various tools and implements comprising an elongated flexible hollow cylindrical sleeve having a first end, a second end, an interior wall surface, and an exterior wall surface; and a lengthwise non-radial slit from the first end to the second end, penetrating through the exterior wall surface through to the interior wall surface.

What is needed is a low cost, compact, and easily removably engagable handle attachment for a tool that provides a comfortable manual grasp that provides a less fatiguing hand hold for the long term or repetitive user.

### SUMMARY OF INVENTION

Broadly, the present invention is a clinician's hand support structure to help provide an ergometric grasp of a syringe housing, wherein the syringe housing is at least partially tapered. The clinician's hand support structure includes a partial discontinuous surrounding sidewall that is about a longitudinal axis, the sidewall having a first end portion and an opposing second end portion along the longitudinal axis, the sidewall also having an outer surface and an inner surface that defines a sidewall interior, further the inner surface having a portion that has a frustroconical shape that is smaller adjacent to the first end portion and larger adjacent to the second end portion. Also, the sidewall has an elongated aperture therethrough that is substantially parallel to the longitudinal axis, wherein the aperture extends from the first end portion to the second end portion, wherein operationally the sidewall inner surface partially creates a frictional encasement of the syringe housing. Further, included is an extension element having a proximal portion and an opposing distal portion forming an extension distance therebetween, with the proximal portion extending from the outer surface, wherein the extension element is positioned to extend radially outward from the longitudinal axis in a plane that extends from the first end portion to the second end portion, wherein operationally the distal portion nests in a palm of a clinician's hand and the sidewall outer surface is grasped by a finger of the clinician.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows a perspective of a clinician's palm side view of the clinician's hand support structure including the partial discontinuous surrounding sidewall, the first end portion of the sidewall, the second end portion of the sidewall, the outer surface of the sidewall, the oppositely disposed sidewall outer surface of the second end portion, the inner surface of the sidewall, and the extension element;

FIG. 2 shows a perspective of a clinician's finger side view of the clinician's hand support structure including the partial discontinuous surrounding sidewall, the first end portion of the sidewall, the second end portion of the sidewall, the outer surface of the sidewall, the oppositely disposed sidewall outer surface of the first and second end portions, the inner surface of the sidewall, the elongated aperture, and the extension element;

FIG. 3 shows a top view of the clinician's hand support structure including the partial discontinuous surrounding sidewall, the second end portion of the sidewall, the outer surface of the sidewall, the oppositely disposed sidewall outer surface of the second end portion, the inner surface of the sidewall, the interior of the sidewall, the cross sectional area of the interior, the elongated aperture, the extension element, and the plane that extends from the first end portion to the second end portion;

FIG. 4 shows an elevation side view of the clinician's hand support structure including the partial discontinuous surrounding sidewall, the first end portion of the sidewall, the second end portion of the sidewall, the outer surface of the sidewall, the oppositely disposed sidewall outer surface of the first and second end portions, the inner surface of the sidewall, the elongated aperture, the extension element, the proximal and distal portions of the extension element, the arcuate margin, the plane that extends from the first end portion to the second end portion, radius of the arcuate margin, coincident positioning of the radius and the maximum distance, termination point of the radius, the origin point of the radius, the dimension between the termination point and the origin, the longitudinal axis of the sidewall, the radial outward extension of the extension element, maximum distance from the first end portion, extension distance between the proximal portion and the distal portion, continuous increasing of the extension distance, area of the elongated aperture, the parallel relationship between the elongated aperture and the longitudinal axis, and the axial separation as between the first end portion and the second end portion;

FIG. 5 shows an elevation side cross sectional view of the clinician's hand support structure including the partial discontinuous surrounding sidewall, the first end portion of the sidewall, the second end portion of the sidewall, the outer surface of the sidewall, the oppositely disposed sidewall outer surface of the first and second end portions, the inner surface of the sidewall, the extension element, the proximal and distal portions of the extension element, the arcuate margin, the plane that extends from the first end portion to the second end portion, the longitudinal axis of the sidewall, radial outward extension of the extension element, maximum distance from the first end portion, extension distance between the proximal portion and the distal portion, and the continuous increasing of the extension distance;

FIG. 6 shows an upper perspective view of the syringe housing;

FIG. 7 shows an upper perspective view of the syringe housing including the partially tapered portion of the housing, the partial frictional encasement as between the sidewall inner surface and the syringe housing, the clinician's hand support structure, the sidewall, the extension, and the removable engagement movement of the syringe housing as against the sidewall inner surface;

FIG. 8 shows a perspective view of the syringe housing including the partially tapered portion of the housing, the partial frictional encasement as between the sidewall inner surface and the syringe housing, the clinician's hand support structure, the sidewall, the extension, and the removable engagement of the syringe housing as against the sidewall inner surface, wherein the clinician's hand support structure is assembled upon the syringe housing;

FIG. 9 shows a side elevation view of the syringe housing including the partially tapered portion of the housing, the partial frictional encasement as between the sidewall inner surface and the syringe housing, the clinician's hand support structure, the sidewall, the extension, and the removable engagement of the syringe housing as against the sidewall inner surface, wherein the clinician's hand support structure is assembled upon the syringe housing;

FIG. 10 shows a perspective view of the syringe housing including the partially tapered portion of the housing, the partial frictional encasement as between the sidewall inner surface and the syringe housing, the clinician's hand support structure, the sidewall, the extension, and the removable engagement of the syringe housing as against the sidewall inner surface, with the clinician's hand support structure assembled upon the syringe housing, wherein the clinician's hand support structure is shown to clear the syringe housing holder; and

FIG. 11 shows a side elevation use view of the clinician's hand support structure assembled upon the syringe housing with the clinician's hand, the clinician's wrist, the lengthwise axis of the clinician's wrist, the palm of the clinician's hand, the profile arc of the palm, the clinician's index fingers, the clinician's thumb, the longwise axis of the clinician's thumb, the nesting of the distal portion in the palm, grasping of the outer surface by the finger, conforming of the arcuate margin to the palm, the substantially coincident relationship of the lengthwise axis and the longwise axis, and the substantial nesting of the clinician's hand support structure within the clinician's palm.

### REFERENCE NUMBERS IN DRAWINGS

50 Clinician's hand support structure
55 Syringe housing
60 Partially tapered portion of the syringe housing 55
65 Partial discontinuous surrounding sidewall
70 Longitudinal axis of the partial discontinuous surrounding sidewall 65
75 First end portion of the partial discontinuous surrounding sidewall 65
80 Second end portion of the partial discontinuous surrounding sidewall 65
85 Outer surface of the partial discontinuous surrounding sidewall 65
86 Oppositely disposed sidewall outer surface of the first end portion 75
87 Oppositely disposed sidewall outer surface of the second end portion 80
90 Inner surface of the partial discontinuous surrounding sidewall 65
95 Interior of the partial discontinuous surrounding sidewall 65
96 Cross sectional area of the interior 95
100 Frustroconical shaped portion of the partial discontinuous surrounding sidewall 65
105 Smaller adjacent frustroconical shaped portion that is adjacent to the frustroconical shaped portion 100
106 Area definition of smaller adjacent frustroconical shaped portion 105
110 Larger adjacent frustroconical shaped portion that is adjacent to the frustroconical shaped portion 100
111 Area definition of larger adjacent frustroconical shaped portion 110
115 Elongated aperture of the partial discontinuous surrounding sidewall 65
116 Area of the elongated aperture 115
120 Substantially parallel relationship as between the elongated aperture 115 to the longitudinal axis 70
125 Partial frictional encasement as between the sidewall inner surface 90 and the syringe housing 55
130 Extension element
135 Proximal portion of the extension element 130
140 Distal portion of the extension element 130
145 Extension distance as between the proximal portion 135 and the distal portion 140
150 Radially outward extension of the extension element 130
155 Plane that extends from the first end portion 75 to the second end portion 80
160 Arcuate margin
165 Continuous increasing of the extension distance 145 along the arcuate margin 160
170 Minimum distance at the second end portion 80 from the arcuate margin 160 to the second end portion 80 oppositely disposed sidewall outer surface 87
175 Maximum distance at the first end portion 75 from the arcuate margin 160 to the first end portion 75 oppositely disposed sidewall outer surface 86
176 Axial separation as between the first end portion 75 and the second end portion 80
180 Length of arcuate margin 160 beyond the first end portion 75 along the longitudinal axis 70
185 Contact surface area as between the arcuate margin 160 and the palm 305
190 Reach of arcuate margin 160 beyond the second end portion 80 along the longitudinal axis 70
195 Radius of the arcuate margin 160
200 Coincident positioning of the radius 195 and the maximum distance 175
205 Termination point of radius 195
210 Origin point of radius 195
215 Dimension between the termination point 205 and the origin point 210
220 Pivotal movement of the radius 195 about the origin 210
300 Clinician's hand
301 Clinician's wrist
302 Lengthwise axis of clinician's wrist
305 Palm of the clinician's hand
306 Profile arc of the palm 305
310 Clinician's index fingers
311 Clinician's thumb
312 Longwise axis of clinician's thumb 311
315 Nesting of the distal portion 140 in the palm 305
320 Grasping of the outer surface 85 by the finger 310
325 Conforming of the arcuate margin 160 to the palm 305
330 Substantially coincident relationship of the lengthwise axis 302 and the longwise axis 312
335 Removable engagement movement of the syringe housing 55 as against the sidewall inner surface 90
340 Substantial nesting of the clinician's hand support structure 50 within the clinician's palm 305
345 Holder for the syringe housing 55

### DETAILED DESCRIPTION

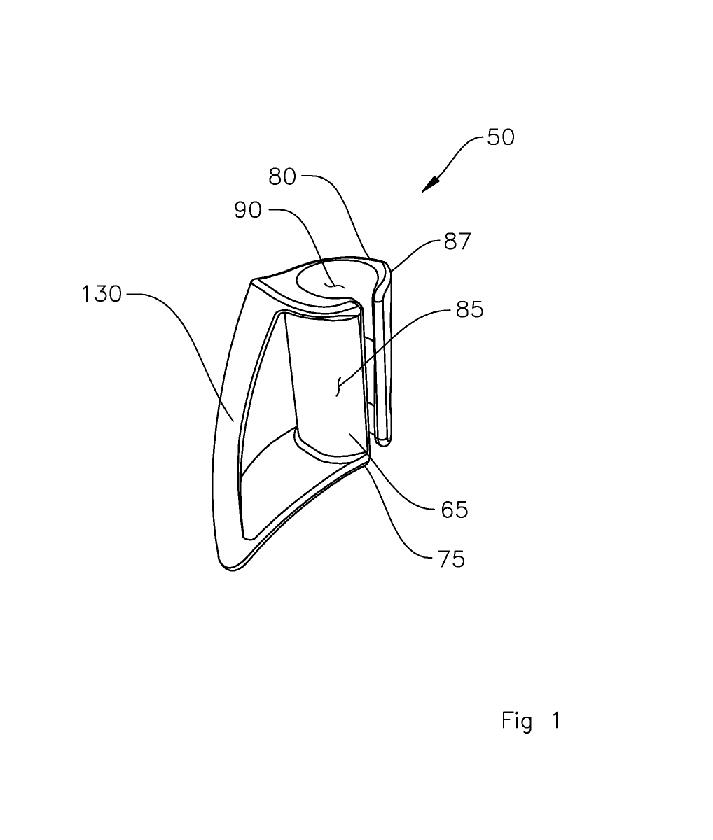

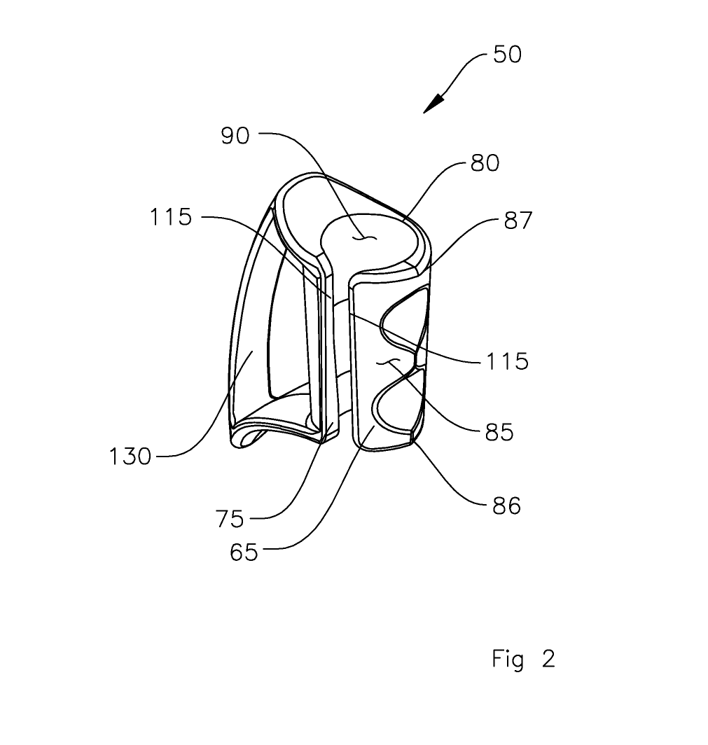

With initial reference to FIG. 1, shown is a perspective of the clinician's palm 305 side view for the clinician's hand support structure 50 including the partial discontinuous surrounding sidewall 65, the first end portion 75 of the sidewall 65, the second end portion 80 of the sidewall 65, the outer surface 85 of the sidewall 65, the oppositely disposed sidewall outer surface 87 of the second end portion 80, the inner surface 90 of the sidewall 65, and the extension element 130. Next, FIG. 2 shows a perspective clinician's finger 310 side view of the clinician's hand support structure 50 including the partial discontinuous surrounding sidewall 65, the first end portion 75 of the sidewall 65, the second end portion 80 of the sidewall 65, the outer surface 85 of the sidewall 65, the oppositely disposed sidewall outer surface 86 of the first portion 75, and the oppositely disposed sidewall outer surface 87 of the second end 80 portion, the inner surface 90 of the sidewall 65, the elongated aperture 115, and the extension element 130.

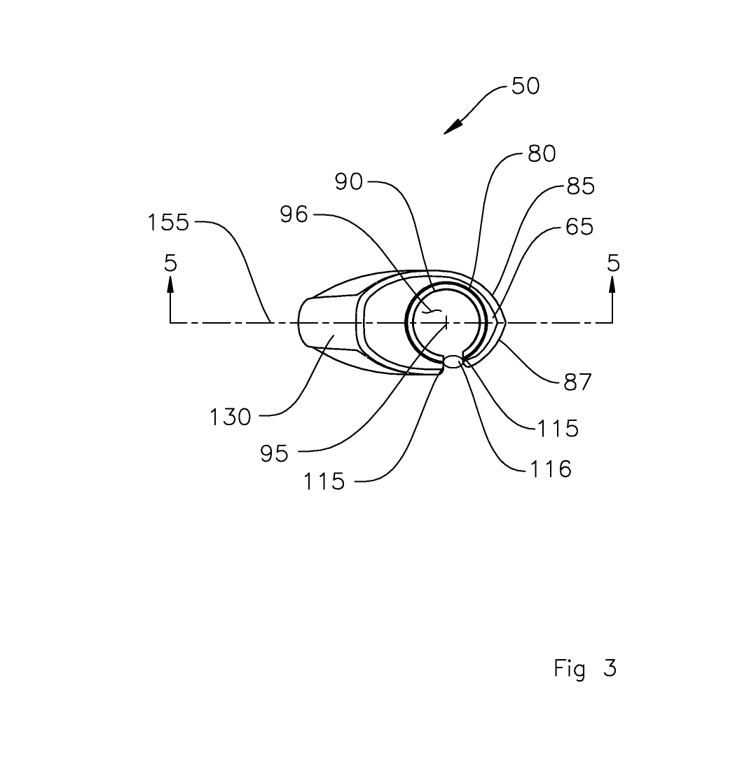

Further, FIG. 3 shows a top view of the clinician's hand support structure 50 including the partial discontinuous surrounding sidewall 65, the second end portion 80 of the sidewall 65, the outer surface 85 of the sidewall 65, the oppositely disposed sidewall outer surface 87 of the second end portion 80, the inner surface 90 of the sidewall 65, the interior 95 of the sidewall 65, the cross sectional area 96 of the interior 95, the elongated aperture 115, the extension element 130, and the plane 155 that extends from the first end portion 75 to the second end portion 80.

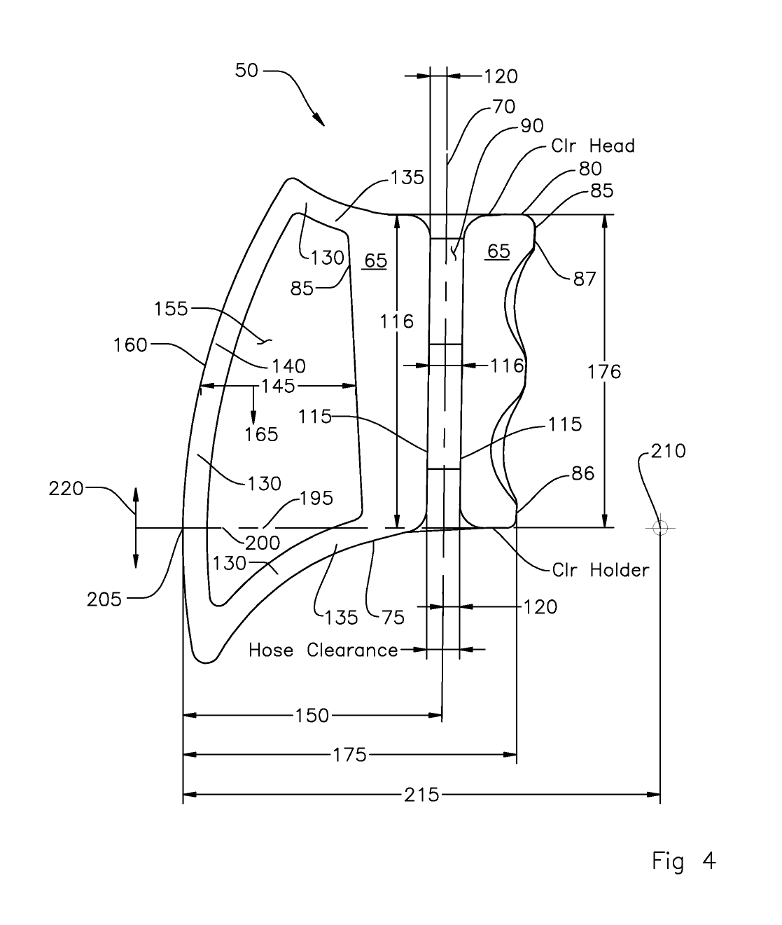

Next, FIG. 4 shows an elevation side view of the clinician's hand support structure 50 including the partial discontinuous surrounding sidewall 65, the first end portion 75 of the sidewall 65, the second end portion 80 of the sidewall 65, the outer surface 85 of the sidewall 65, the oppositely disposed sidewall outer surface 86 of the first end portion 75 of the sidewall 65, and the oppositely disposed sidewall outer surface 87 of the second end portion 80 of the sidewall 65, the inner surface 90 of the sidewall 65. Also, FIG. 4 shows the elongated aperture 115, the extension element 130, the proximal 135 and distal 140 portions of the extension element 130, the arcuate margin 160, the plane 155 that extends from the first end portion 75 to the second end portion 80, radius 195 of the arcuate margin 160, coincident positioning 200 of the radius 195 and the maximum distance 175, termination point 205 of the radius 195, the origin point 210 of the radius 195, and the dimension 215 between the termination point 205 and the origin 210. Further, FIG. 4 shows the longitudinal axis 70 of the sidewall 65, radial outward extension 150 of the extension element 130, maximum distance 175 from the first end portion 75, extension distance 145 between the proximal portion 135 and the distal portion 140, continuous increasing 165 of the extension distance 145, area 116 of the elongated aperture 115, the substantially parallel relationship 120 between the elongated aperture 115 and the longitudinal axis 70, and the axial separation 176 as between the first end portion 75 and the second end portion 80.

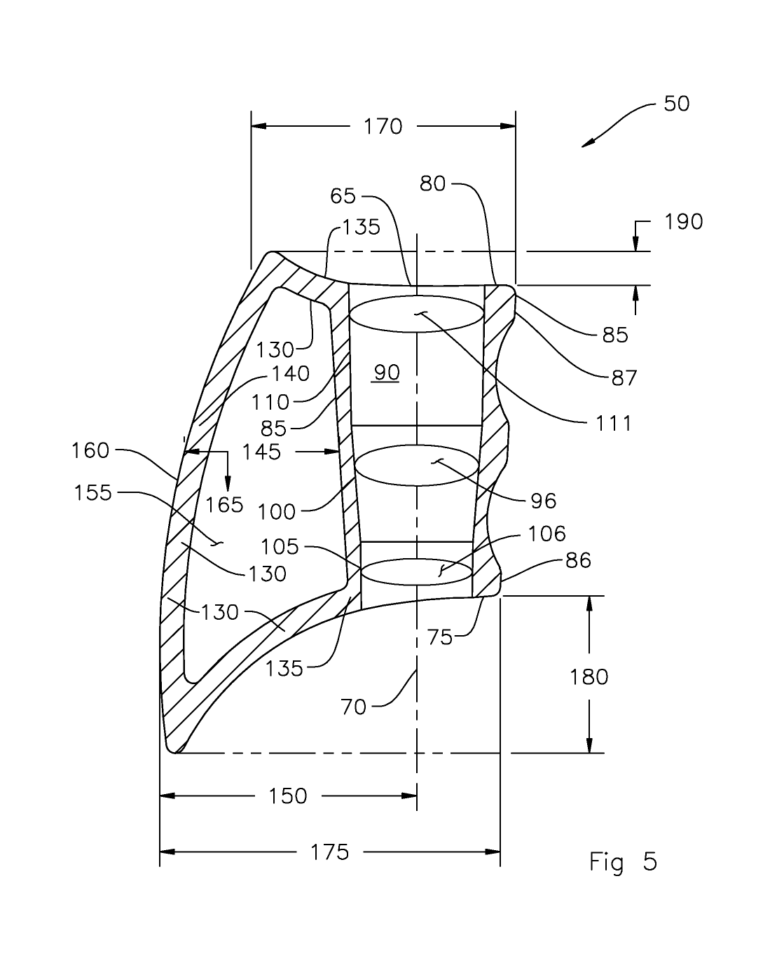

Continuing, FIG. 5 shows an elevation side cross sectional view of the clinician's hand support structure 50 including the partial discontinuous surrounding sidewall 65, the first end portion 75 of the sidewall 65, the second end portion 80 of the sidewall 65, the outer surface 85 of the sidewall 65, the oppositely disposed sidewall outer surface 86 of the first end portion 75, and the oppositely disposed sidewall outer surface 87 of the second end portion 80, and the inner surface 90 of the sidewall 65. Further shown in FIG. 5 is the extension element 130, the proximal 135 and distal 140 portions of the extension element 130, the arcuate margin 160, the plane 155 that extends from the first end portion 75 to the second end portion 80, longitudinal axis 70 of the sidewall 65, radial outward extension 150 of the extension element 130, maximum distance 175 from the first end portion 75, extension distance 145 between the proximal portion 135 and the distal portion 140, and the continuous increasing 165 of the extension distance 145.

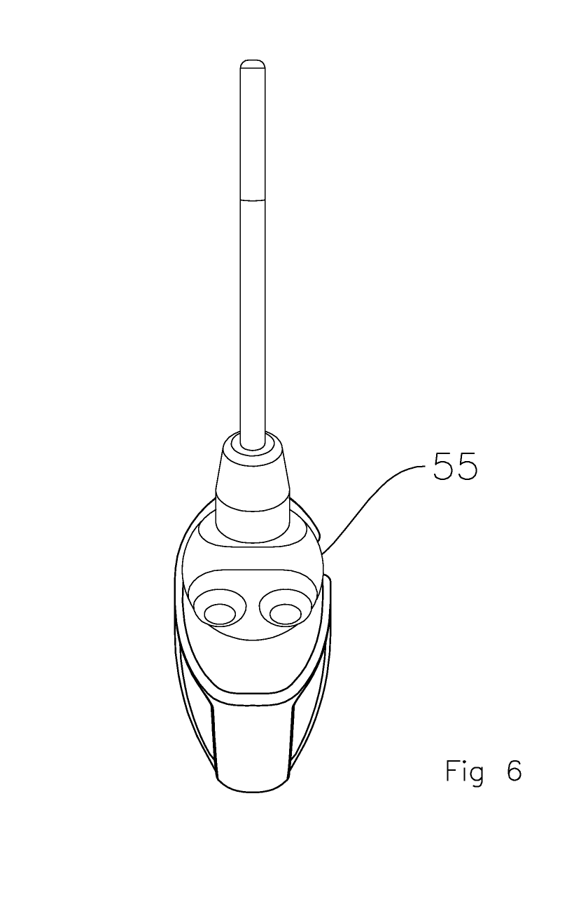

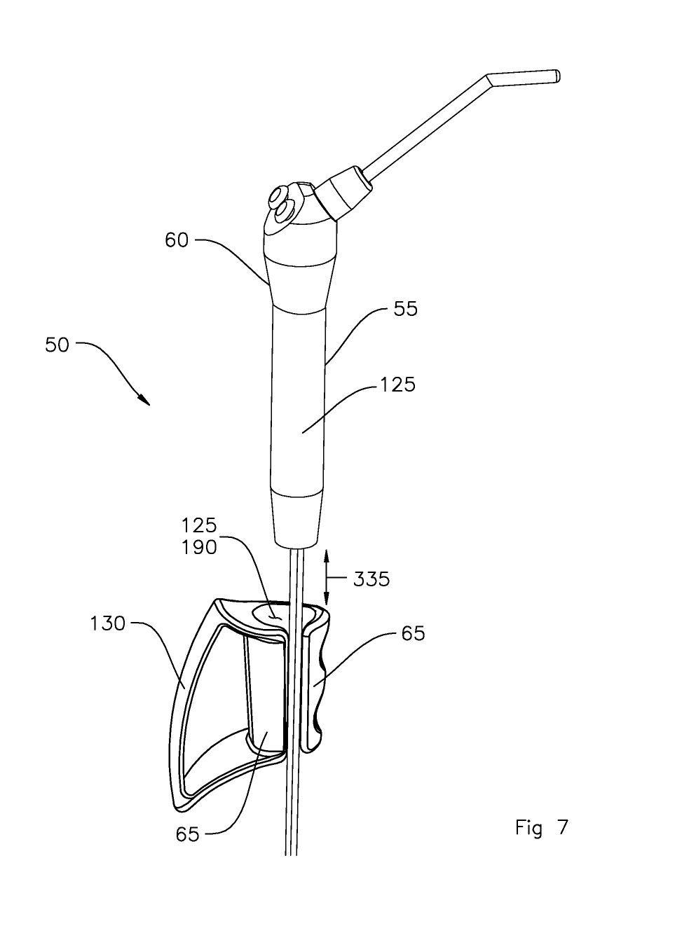

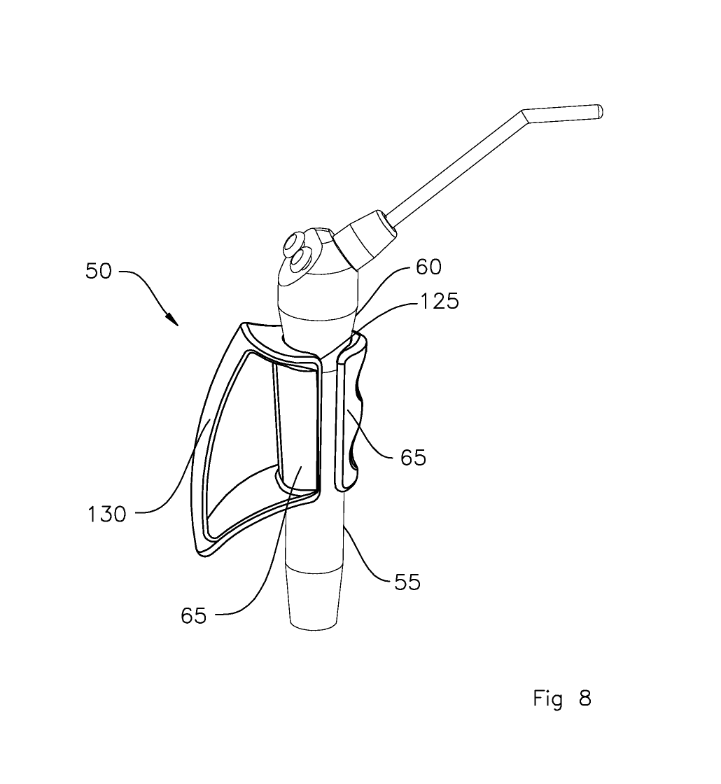

Further, FIG. 6 shows an upper perspective view of the syringe housing 55. Next, FIG. 7 shows an upper perspective view of the syringe housing 55 including the partially tapered portion 60 of the housing 55, the partial frictional encasement as between the sidewall 65 inner surface 90 and the syringe housing 55, the clinician's hand support structure 50, the sidewall 65, the extension 130, and the removable engagement movement 335 of the syringe housing 55 as against the sidewall 65 inner surface 90. Next, FIG. 8 shows a perspective view of the syringe housing 55 including the partially tapered portion 60 of the housing 55, the partial frictional encasement as between the sidewall 65 inner surface 90 and the syringe housing 55, the clinician's hand support structure 50, the sidewall 65, the extension 130, and the removable engagement of the syringe housing 55 as against the sidewall 65 inner surface 90, wherein the clinician's hand support structure 50 is assembled upon the syringe housing 55.

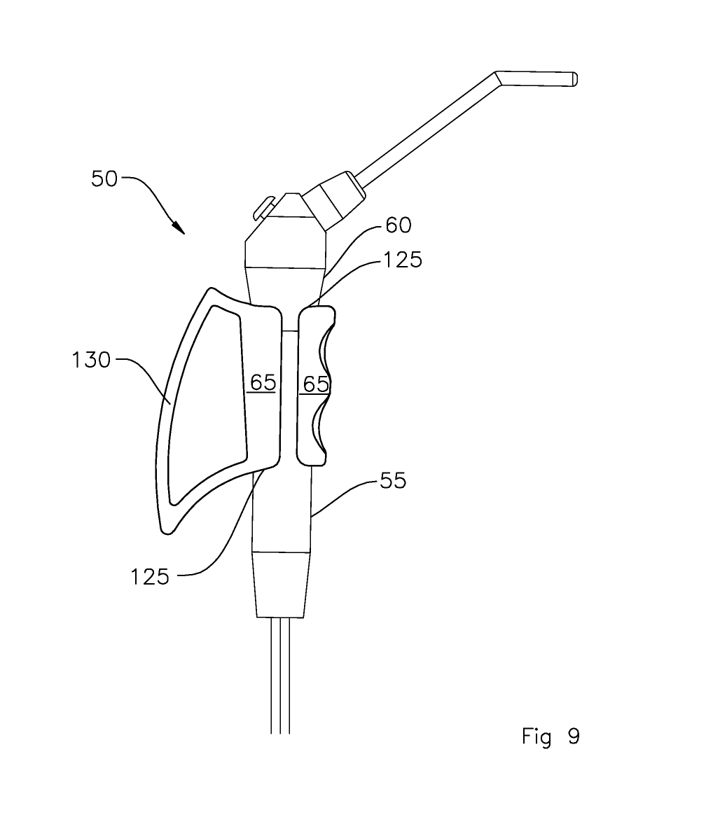

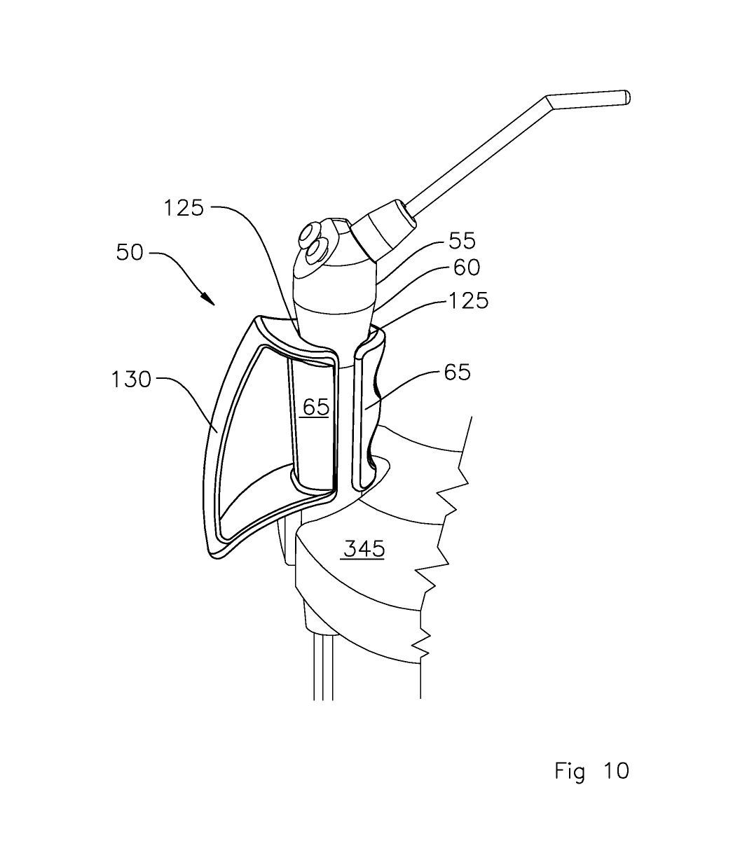

Continuing, FIG. 9 shows a side elevation view of the syringe housing 55 including the partially tapered portion 60 of the housing 55, the partial frictional encasement as between the sidewall 65 inner surface 90 and the syringe housing 55, the clinician's hand support structure 50, the sidewall 65, the extension 130, and the removable engagement of the syringe housing 55 as against the sidewall 65 inner surface 90, wherein the clinician's hand support structure 50 is assembled upon the syringe housing 55. Next, FIG. 10 shows a perspective view of the syringe housing 55 including the partially tapered portion 60 of the housing 55, the partial frictional encasement as between the sidewall 65 inner surface 90 and the syringe housing 55, the clinician's hand support structure 50, the sidewall 65, the extension 130, and the removable engagement of the syringe housing 55 as against the sidewall 65 inner surface 90, wherein the clinician's hand support structure 50 is assembled upon the syringe housing 55, wherein the clinician's hand support structure 50 is shown to clear the syringe housing 55 holder 345.

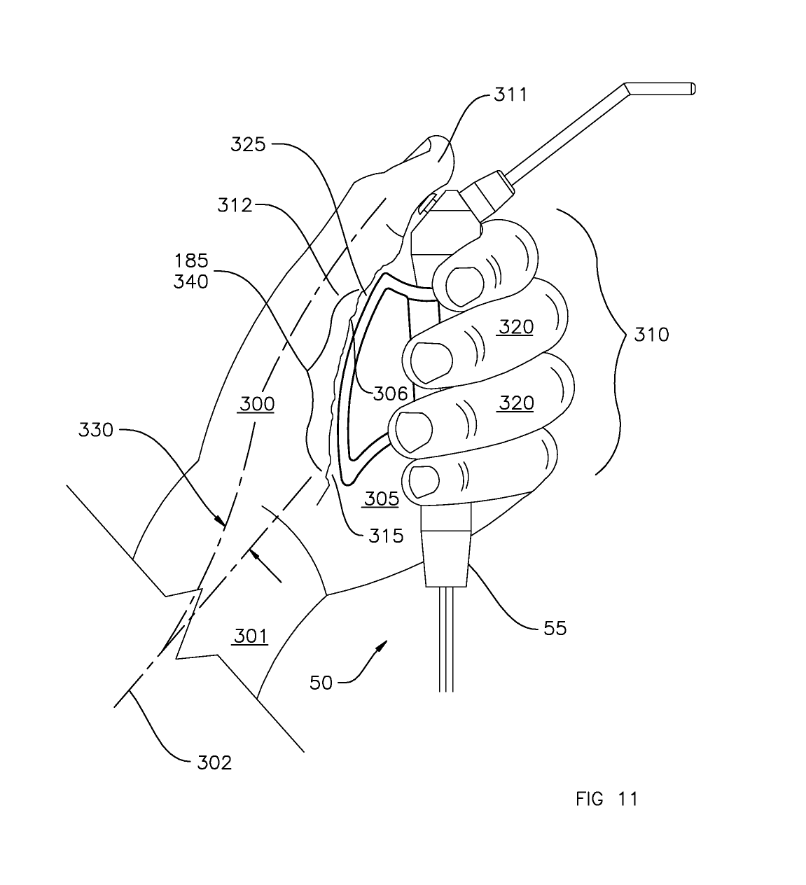

Further, FIG. 11 shows a side elevation use view of the clinician's hand support structure 50 assembled upon the syringe housing 55 with the clinician's hand 300, the clinician's wrist 301, the lengthwise axis 302 of the clinician's wrist 301, the palm 305 of the clinician's hand 300, the profile arc 306 of the palm 305, the clinician's index fingers 310, the clinician's thumb 311, the longwise axis 312 of the clinician's thumb 311, the nesting 315 of the distal portion 140 of the palm 305, grasping 320 of the outer surface 85 by the finger 310, conforming 325 of the arcuate margin 160 to the palm 305, the substantially coincident relationship 330 of the lengthwise axis 302 and the longwise axis 312, and the substantial nesting 340 of the clinician's hand support structure 50 within the clinician's palm 305.

Broadly, in looking at FIGS. 1 through 5 for the present invention of the clinician's hand support structure 50 which is to help provide an ergometric grasp of a syringe housing, as best shown in FIGS. 7 through 11, wherein the syringe housing 55 is at least partially tapered 60, see in particular FIG. 7. The clinician's hand support structure 50 includes the partial discontinuous surrounding sidewall 65 that is about a longitudinal axis 70, see FIGS. 4 and 5, the sidewall 65 having a first end portion 75 and the opposing second end portion 80 along the longitudinal axis 70, the sidewall 65 also having an outer surface 85 and an inner surface 90 that defines a sidewall 65 interior 95, see FIGS. 1 through 5. Further, the inner surface 90 having a portion that has a frustroconical shape 100 that is smaller adjacent 105 to the first end portion 75 and larger adjacent 110 to the second end portion 80, see FIGS. 4 and 5. Also, the sidewall 65 has an elongated aperture 115 therethrough that is substantially parallel 120 to the longitudinal axis 70, wherein the aperture 115 extends from the first end portion 75 to the second end portion 80, see FIGS. 2, 3, and 4 for detail. Wherein, operationally the sidewall 65 inner surface 90 partially creates a frictional encasement of the syringe housing 55, as best shown in FIGS. 7 and 8. Further, included is an extension element 130 having the proximal portion 135 and the opposing distal portion 140 forming an extension distance 145 therebetween, with the proximal portion 135 extending from the outer surface 85, wherein the extension element 130 is positioned to extend radially outward 150 from the longitudinal axis 70 in the plane 155 that extends from the first end portion 75 to the second end portion 80, as best shown in FIGS. 1 through 5. Wherein operationally, the distal portion 140 nests 315 in the palm 305 of a clinician's hand 300 and the sidewall 65 outer surface 85 is grasped by a finger 310 of the clinician, as best shown in FIG. 11.

Further on the clinician's hand support structure 50, concerning the elongated aperture 115, wherein the elongated aperture 115 has an area 116 that is less than a cross sectional area 96 of the interior 95, see FIGS. 2, 3, and 4, and FIGS. 7 and 8. Wherein preferably the elongated aperture 115 area 116 is about 0.34 inches squared and preferably the area 96 is about 0.36 inches squared. This is to operationally help facilitate a removable engagement of the syringe housing 55 encasement upon the sidewall 65 inner surface 90, see FIGS. 7, 8, and 9, and in particular FIG. 7, that shows the insertion of the syringe housing 55 into the interior 95 as against the inner surface 90 for the partial frictional encasement 125 as shown in FIG. 8, thus the area 116 must allow for syringe 55 removable engagement movement 335 clearance while still encasing 125 and retaining the syringe housing 55 for the use as shown in FIG. 11. The area 116 provides access for the removable engagement movement 335 while leaving enough area 96 for the encasement 125, see FIGS. 7 and 8.

Continuing on the clinician's hand support structure 50, relating to the frustroconical shape 100, the smaller frustroconical shape 105 is defined as an area 106 that is no greater than ninety five percent (95%) of the larger frustroconical shape 110 defined as an area 111, as best shown in FIG. 5, being operational to create the frictional encasement 125 as between the syringe housing 55 and the frustroconical shape 100, which includes the smaller frustroconical shape 105 and the larger frustroconical shape 110, as best shown in FIGS. 7 and 8, thus facilitating the use shown in FIG. 11, with the syringe 55 frictionally encased 125 into the frustroconical shape 100 to secure the hand support structure 50 to the syringe 55. The preferred area 106 is about 0.28 inches squared and the preferred area 111 is about 0.44 inches squared. Thus accommodating a more relaxed and ergometric and hand 300 grip via providing more gripping area and a wider grip-as between the palm 305 and fingers 310, both through the extension element 130, particularly the arcuate margin 160 nesting 315 in the palm 305 with an opposing wider hand 300 grip via the fingers 310 gripping as against the sidewall 65 outer surface 85, resulting in the wider distance for gripping as between the clinician's palm 305 and the clinician's fingers 310.

Next, for the clinician's hand support structure 50, in relation to the extension element 130, the distal portion 140 terminates in an arcuate margin 160 that is defined by continuously increasing 165 the extension distance 145 in going from the second end portion 80 to the first end portion 75 thus defining a minimum distance 170 at the second end portion 80 from the arcuate margin 160 to an oppositely disposed sidewall 65 outer surface 87 to a maximum distance 175 at the first end portion 75 from the arcuate margin 160 to an oppositely disposed sidewall 65 outer surface 86, see FIGS. 4 and 5. Preferably the extension distance 145 goes from distance 170 of about 1.5 inches to distance 175 of about 2.1 inches. This is to operationally have the arcuate margin 160 approximately conform 325 to a palm 305 arc of the clinician's hand 300, as best shown in FIG. 11, thus resulting in an approximately even pressure of the arcuate margin 160 as against the palm 305 for minimal fatigue.

Continuing on the arcuate margin 106 for the clinician's hand support structure 50, the arcuate margin 160 preferably extends a length 180 beyond the first end portion 75 along the longitudinal axis 70, as shown in FIG. 5, being preferably in the range of about 0.9 inches, to operationally increase a contact surface area 185 as between the arcuate margin 160 and the palm 305 arc 325, as best shown in FIG. 11, thus resulting in the preferred contact area 185 of the arcuate margin being about 1.5 inches squared. Further, on the clinician's hand support structure 50, the arcuate margin 160 extends a reach distance 190 beyond the second end portion 80 along the longitudinal axis 70, as best shown in FIGS. 4 and 5, wherein preferably reach 190 is about 0.3 inches. Again this is to operationally increase the contact surface area 185 as between the arcuate margin 160 and the palm 305 arc 325, with the preferred contact area 185 being about 1.5 inches squared.

Continuing, for the clinician's hand support structure 50, wherein the minimum distance 170 is less than an axial separation 176 as between the first end portion 75 and the second end portion 80 along the longitudinal axis 70, as best shown in FIGS. 4 and 5, wherein the axial separation 176 is preferably about 1.7 inches. The purpose of this is to operationally help provide for the clinician's thumb 311 longwise axis 312 to be substantially coincident 330 to a clinician's wrist 301 lengthwise axis 302 to help minimize the fatigue of the clinician's hand 300, as best shown in FIG. 11, thus not causing the thumb axis 312 to be at a significant displaced angle to the axis 302 that could increase stress and fatigue in the clinician's wrist 301 and hand 300.

Also, on the clinician's hand support structure 50, wherein the axial separation 176 is less than the maximum distance 175, as shown in FIG. 4, to operationally keep the clinician's hand support structure 50 substantially nested 315 within the clinician's palm 305, as best shown in FIG. 11. This facilitates the clinician's fingers 310 to “cradle” the outer surface 85, thus resisting movement along the axis 70-to further secure the nesting 315 of the arcuate margin 160 in the palm 305-for a secure, relaxed, and comfortable grasp of the clinician's hand 300 upon the hand support structure 50 and ultimately supporting the syringe 55.

Further, on the clinician's hand support structure 50, wherein the arcuate margin 160 is further defined by a radius 195 that is positioned coincident to the maximum distance 175 wherein the radius 195 has a termination point 205 at the arcuate margin 160 and an opposing origin point 210 at a dimension 215 from the termination point 205, wherein the origin point 210 is positioned opposite of the extension element 130, wherein also the radius 195 termination point 205 when pivoted 220 about the origin point 210, form the arcuate margin 150, as best shown in FIG. 4. Thus, to operationally match the clinician's palm 305 profile 306 for conforming 325 nesting 315, as best shown in FIG. 11. The radius 195 is preferably about 5.6 inches which would also equal the dimension 215 being the distance as between the termination point 205 and the origin point 210, see FIG. 4. In addition, for the clinician's hand support structure 50, wherein the radius dimension 195 is at least two times the maximum distance 175, thus providing that the arcuate margin 160 with a radius 195 and area 185 that provide for comfortable substantial nesting 340 of the arcuate margin 160 to operationally substantially match the clinician's palm 305 profile 306 as shown in FIGS. 4 and 11.

### CONCLUSION

Accordingly, the present invention of an clinician's hand support structure has been described with some degree of particularity directed to the embodiments of the present invention. It should be appreciated, though, that the present invention is defined by the following claims construed in light of the prior art so modifications or changes may be made to the exemplary embodiments of the present invention without departing from the inventive concepts contained therein.

1. A clinician's hand support structure to help provide an ergometric grasp of a syringe housing, wherein said syringe housing is at least partially tapered, comprising:
(a) a partial discontinuous surrounding sidewall that is about a longitudinal axis, said sidewall having a first end portion and an opposing second end portion along said longitudinal axis, said sidewall having an outer surface and an inner surface that defines a sidewall interior, said inner surface having a portion that has a frustroconical shape that is smaller adjacent to said first end portion and larger adjacent to said second end portion, said sidewall having an elongated aperture therethrough that is substantially parallel to said longitudinal axis, wherein said aperture extends from said first end portion to said second end portion, wherein operationally said sidewall inner surface partially creates a frictional encasement of the syringe housing; and
(b) an extension element having a proximal portion and an opposing distal portion forming an extension distance therebetween, said proximal portion extending from said outer surface, wherein said extension element is positioned to extend radially outward from said longitudinal axis in a plane that extends from said first end portion to said second end portion, wherein operationally said distal portion nests in a palm of a clinician's hand and said sidewall outer surface is grasped by a finger of the clinician.

2. A clinician's hand support structure according to claim 1 wherein said elongated aperture has an area that is less than a cross sectional area of said interior, to operationally help facilitate a removable engagement of the syringe housing encasement upon the sidewall inner surface.

3. A clinician's hand support structure according to claim 1 wherein said smaller frustroconical shape defined as an area is no greater than ninety five percent (95%) of said larger frustroconical shape defined as an area, being operational to create said frictional encasement.

4. A clinician's hand support structure according to claim 1 wherein said distal portion terminates in an arcuate margin that is defined by continuously increasing said extension distance in going from said second end portion to said first end portion thus defining a minimum distance at said second end portion from said arcuate margin to an oppositely disposed sidewall outer surface to a maximum distance at said first end portion from said arcuate margin to an oppositely disposed sidewall outer surface to operationally have said arcuate margin approximately conform to a palm arc of the clinician's hand.

5. A clinician's hand support structure according to claim 4 wherein said arcuate margin extends a length beyond said first end portion along said longitudinal axis to operationally increase a contact surface area as between said arcuate margin and the palm arc.

6. A clinician's hand support structure according to claim 4 wherein said arcuate margin extends a reach beyond said second end portion along said longitudinal axis to operationally increase a contact surface area as between said arcuate margin and the palm arc.

7. A clinician's hand support structure according to claim 4 wherein said minimum distance is less than an axial separation as between said first end portion and said second end portion along said longitudinal axis, to operationally help provide for the clinician's thumb longwise axis to be substantially coincident to a clinician's wrist lengthwise axis to help minimize the fatigue of the clinician's hand.

8. A clinician's hand support structure according to claim 7 wherein said axial separation is less than said maximum distance to operationally keep said clinician's hand support structure substantially nested within the clinician's palm.

9. A clinician's hand support structure according to claim 4 wherein said arcuate margin is further defined by a radius that is positioned coincident to said maximum distance, wherein said radius has a termination point at said arcuate margin and an opposing origin point at a dimension from said termination point, wherein said origin point is positioned opposite of said extension element, said radius termination point when pivoted about said origin point forms said arcuate margin, to operationally match the clinician's palm profile.

10. A clinician's hand support structure according to claim 9 wherein said radius dimension is at least two times said maximum distance, to operationally substantially match the clinician's palm profile.

11. A clinician's hand support structure to help provide an ergometric grasp of a syringe housing, wherein said syringe housing is at least partially tapered, comprising:
(a) a partial discontinuous surrounding sidewall that is about a longitudinal axis, said sidewall having a first end portion and an opposing second end portion along said longitudinal axis, said sidewall having an outer surface and an inner surface that defines a sidewall interior, said inner surface having a portion that has a frustroconical shape that is smaller adjacent to said first end portion and larger adjacent to said second end portion, said sidewall having an elongated aperture therethrough that is substantially parallel to said longitudinal axis, wherein said aperture extends from said first end portion to said second end portion, wherein operationally said sidewall inner surface partially creates a frictional encasement of the syringe housing; and
(b) an extension element having a proximal portion and an opposing distal portion forming an extension distance therebetween, said proximal portion extending from said outer surface, wherein said extension element is positioned to extend radially outward from said longitudinal axis in a plane that extends from said first end portion to said second end portion, wherein said distal portion terminates in an arcuate margin that is defined by continuously increasing said extension distance in going from said second end portion to said first end portion thus defining a minimum distance at said second end portion from said arcuate margin to an oppositely disposed sidewall outer surface to a maximum distance at said first end portion from said arcuate margin to an oppositely disposed sidewall outer surface, wherein operationally said distal portion nests in a palm of a clinician's hand, wherein said arcuate margin approximately conforms to a palm arc of the clinician's hand and said sidewall outer surface is grasped by a finger of the clinician.

12. A clinician's hand support structure according to claim 11 wherein said minimum distance is less than an axial separation as between said first end portion and said second end portion along said longitudinal axis, to operationally help provide for the clinician's thumb longwise axis to be substantially coincident to a clinician's wrist lengthwise axis to help minimize the fatigue of the clinician's hand.

13. A clinician's hand support structure according to claim 12 wherein said axial separation is less than said maximum distance to operationally keep said clinician's hand support structure substantially nested within the clinician's palm.

14. A clinician's hand support structure according to claim 11 wherein said arcuate margin is further defined by a radius that is positioned coincident to said maximum distance wherein said radius has a termination point at said arcuate margin and an opposing origin point at a dimension from said termination point, wherein said origin point is positioned opposite of said extension element, said radius termination point when pivoted about said origin point forms said arcuate margin, to operationally match the clinician's palm profile.

15. A clinician's hand support structure according to claim 14 wherein said radius dimension is at least two times said maximum distance, to operationally substantially match the clinician's palm profile.
